(19) United States
(12) Patent Application Publication
(10) Pub. No.: US 2015/0265140 A1
(43) Pub. Date: Sep. 24, 2015

(54) INSTRUMENT HOLDER FOR MOUNTING A MEDICAL INSTRUMENT ON A JOINT ARM

(71) Applicant: MAQUET GMBH, Rastatt (DE)

(72) Inventors: Dieter Hafner, Offenburg (DE); Ulrich Wyslucha, Weingarten (DE)

(21) Appl. No.: 14/440,974

(22) PCT Filed: Oct. 21, 2013
(86) PCT No.: PCT/EP2013/071948

§ 371 Date: May 6, 2015

(30) Foreign Application Priority Data
Nov. 9, 2012 (DE) 10 2012 110 766.1

(51) Int. Cl. A61B 1/00

(52) U.S. Cl. CPC A61B 1/00149

(57) ABSTRACT

An instrument holder (50) is described for mounting a medical instrument (102) on a joint arm (10), said instrument holder comprising a housing (56), a holder formed on the housing (56) in order to hold the medical instrument (102), and a coupling device (52) formed on the housing (56) in order to attach the housing (56) to the joint arm (10). The holder has a receiving groove (80) adapted to a part (104) of the medical instrument (102), in which receiving groove (80) this instrument part (104) is receivable, and an elastic pull strap (82) that is attachable to the housing (56) in such a manner that it surrounds the receiving groove (80) together with the instrument part (104) arranged in the receiving groove (80) in order to hold the instrument (102).

### CROSS REFERENCE TO RELATED APPLICATIONS

This application is entitled to the benefit of and incorporates by reference subject matter disclosed in International Patent Application No. PCT/EP2013/071948 filed on Oct. 21, 2013 and German Patent Application No. 10 2012 110 766.1 filed Nov. 9, 2012.

### TECHNICAL FIELD

The present invention relates to an instrument holder for mounting a medical instrument, in particular an endoscope, on a joint arm, comprising a housing, a holder formed on the housing in order to hold the medical instrument, and a coupling device formed on the housing in order to attach the housing to the joint arm.

### BACKGROUND

For surgical applications nowadays increasingly assistance systems are used comprising a joint arm, which is for example attachable to the slide rail of an operating table. A medical instrument is coupled to the free end of the freely movable joint arm. For example, in laparoscopy, a rigid endoscope provided with an optical system is mounted on the joint arm and oriented such that the target region to be examined or to be treated surgically can be seen into by the surgeon.

In order to couple different instruments selectively with the joint arm, such assistance systems often include an instrument holder as mechanical interface between instrument and joint arm. Such an instrument holder includes a coupling device, which is attached to the free end of the joint arm, as well as a holder for the medical instrument. The holder is for example formed as screw clamp and has to be operated by the surgeon or an assistant with both hands in order to attach the medical instrument to the instrument holder.

### SUMMARY

It is the object of the invention to provide an instrument holder which has a simple design and can be easily operated.

The invention solves this object by the instrument holder for mounting a medical instrument on a joint arm, comprising: a housing, a holder formed on the housing in order to hold the medical instrument, and a coupling device formed on the housing in order to attach the housing to the joint arm, wherein the holder has a receiving groove adapted to a part of the medical instrument, in which receiving groove said instrument part is receivable, and an elastic pull strap that is attachable to the housing in such a manner that it surrounds the receiving groove together with the instrument part arranged in the receiving groove in order to hold the instrument.

According to the invention, the holder has a receiving groove adapted to a part of the medical instrument, in which receiving groove this instrument part is receivable, and an elastic pull strap, which is attachable to the housing in such a manner that it surrounds the receiving groove together with the instrument part arranged in the receiving groove in order to hold the instrument.

The use of a an elastic pull strap which clamps the instrument in the receiving groove has the advantage over conventionally used screw clamps that one can readjust the instrument attached to the instrument holder within certain limits without having to detach the instrument from the instrument holder. For example, it is possible to rotate the instrument about its longitudinal axis without opening the pull strap. The instrument holder according to the invention thus allows, in addition to the required stability with which the instrument is to be held, also a certain flexibility of the instrument movement and thus simplifies the handling significantly.

The housing of the instrument holder on which on the one hand the holder for the medical instrument and on the other hand the coupling device for attaching the instrument holder to the joint arm are formed, for example can be produced cost-effectively in the form of a plastic injection-moulded article. Thus, it is possible to design the instrument holder according to the invention as sterile disposable.

In order to attach the medical instrument to the instrument holder coupled with the joint arm, the user only has to arrange the instrument in the receiving groove and then has to pull the pull strap, using its elasticity, over the receiving groove and to mount it in a suitable manner on the housing of the instrument holder. For this, he requires only hand. The same applies to detaching of the instrument from the instrument holder. The invention thus allows for a particularly simple one-hand operation of the instrument holder.

Preferably, the pull strap has a first strap portion, which is attached on one side of the receiving groove, and a second strap portion, which is mountable on the other side of the receiving groove on the housing under elastic expansion on the pull strap, whereby a third strap portion positioned between the first and the second strap portion surrounds the receiving groove in an elastically expanded state. In a particularly preferred embodiment, the pull strap thus surrounds the receiving groove transversely to the longitudinal extension thereof, wherein the first strap portion is fixed on one side and the second strap portion is fixed on the other side of the receiving groove. The portion of the instrument, which is fitted into the receiving groove, is thus particularly reliably locked.

The second strap portion is preferably mountable via a pin/hole-connection on the housing. Thus, it is possible to pull the pull strap in a particularly simple manner over the receiving groove and to clamp it on the housing of the instrument holder. Also the first strap portion of the pull strap can be mounted via a corresponding pin/hole-connection on the housing.

In an advantageous embodiment, the second strap portion has a mounting hole and the housing has a mounting projection engaging the mounting hole, which mounting projection has a locking nose at its free end pointing away from the receiving groove. Consequently, the locking nose points into a direction which acts against the tension generated by the elastic deformation of the pull strap. This ensures that the second strap portion of the pull strap does not slip from the mounting projection.

A corresponding embodiment is also advantageous for attaching the first strap portion on the other side of the receiving groove. In this case, the pull strap is detachable in its entirety from the housing. Alternatively, it is also possible to attach the first strap portion firmly to the housing, so that the second strap portion constitutes the free end of a pull strap firmly attached to the housing.

Preferably, the first, the second and the third strap portion form a straight, elongated element in the untensioned state of the pull strap. Thereby the width of the elongated element is preferably equal to the length of the receiving groove over which the pull strap extends for holding the medical instrument.

In a particularly preferred embodiment, the pull strap has a fourth strap portion adjacent to the third strap portion, which fourth strap portion projects in the untensioned state of the pull strap at an angle, preferably at a right angle, from the straight element. This fourth strap portion forms a handle portion, at which the user grabs the pull strap in order to pull it over the receiving groove. Preferably, the surface of the fourth strap portion is grooved or surface-treated in another manner in order to prevent slipping of the user's fingers from the pull strap.

The pull strap is preferably formed from an elastomer, e.g. from rubber or silicon. Any kind of materials can be used for the pull strap, provided that the latter has the desired elasticity. Thereby the elasticity of the pull strap is for example in a shore hardness range of 20 to 100, preferably in a shore hardness range of 30 to 90 and particularly preferred in a shore hardness range of 45 to 75. The material thickness of the pull strap is for example in a range of 1 to 12 mm, preferably in a range of 2 to 10 mm and particularly preferred in a range of 3 to 7 mm. Relevant for the measurement of the elasticity of the pull strap is that the pull strap is tensible with the force of one hand.

The receiving groove is preferably trapezoid in cross section. Such a trapezium shape is particularly well suited for holding the medical instrument as it as it has a self-centering and tolerance-compensating effect. Of course, the receiving groove is however not limited to a trapezoid cross section. Many different shapes are possible for the receiving groove. It is advantageous, if the instrument clamped in the receiving groove is pressed against two walls tapered in the direction of the groove bottom. Due to the wedge effect realized in this manner a great clamping effect can be achieved with a small clamping force. This is for example also possible with a triangular cross section or a half-ellipsoidal cross section.

In a particularly preferred embodiment, the receiving groove is formed in an elastic receiving piece, which rests in a fitting opening formed in the housing. The fitting opening provides the possibility to attach different receiving pieces for different medical instruments, which differ for example in their cross section shape, in particular their diameter, as instrument holder. The instrument holder is thus usable for different instruments.

As the receiving piece as well as the pull strap are formed elastically, it allows a certain flexibility in the orientation of the medical instrument attached to the instrument holder without having to move the joint arm itself. The elasticity of the receiving piece thereby is for example in a shore hardness range of 20 to 100, preferably in a shore hardness range of 30 to 90 and particularly preferred in a shore hardness range of 50 to 75. The softer the receiving piece is, the more likely the receiving piece has a certain “adhesive effect”, which favors the fixing of the instrument in the receiving groove.

Preferably, the receiving piece and the fitting opening engage in a dovetail joint manner. A dovetail joint allows for a particularly reliable attachment of the receiving piece in the fitting opening.

In a particularly preferred embodiment, the housing of the instrument holder is rotatable relative to the coupling device about a rotational axis. This allows for a rotation of the instrument attached to the instrument holder without having to adjust the joint arm.

In a preferred embodiment, the coupling device comprises a locking plate with a first toothing, which is in locking engagement with a second toothing formed on the housing, wherein the second toothing is detachable from the first toothing against the biasing force exerted by a biasing element along the rotational axis in order to rotate the housing relative to the locking plate about the rotational axis. The two toothings can for example be formed such that the housing can be rotated in predetermined angle intervals relative to the locking plate.

Preferably, the instrument holder is formed such that is sterilizable. In this case, the instrument holder is preferably a disposable. However, it is also possible to design the instrument holder for multiple use, in particular sterilizable multiple times.

According to a further aspect of the invention, an assistance system is provided comprising a joint arm, a medical instrument and an instrument holder of the above-described type for mounting the medical instrument on the joint arm. The medical instrument is for example an endoscope, which is used in laparoscopy, to give the surgeon the possibility to see into the target region to be treated.

### BRIEF DESCRIPTION OF THE DRAWINGS

The invention will be explained in more detail in the following on the basis of the Figures, wherein:

FIG. 1 shows a joint arm, which is part of the assistance system according to the invention;

FIG. 2 shows an instrument holder according to the invention, which is to be mounted on the joint arm according to FIG. 1;

FIG. 3 shows an exploded view of the instrument holder;

FIG. 4 shows a perspective view of the housing of the instrument holder;

FIG. 5 shows a further perspective view of the housing of the instrument holder;

FIG. 6 shows a view of the assembled housing;

FIG. 7 shows a view of the assembled housing with another receiving piece;

FIG. 8 shows an illustration showing how the housing of the instrument holder is rotated relative to the locking plate;

FIG. 9 shows an illustration showing how the pull strap is positioned around an endoscope; and

FIG. 10 shows an illustration showing how the endoscope is rotatably held by means of the pull strap.

### DETAILED DESCRIPTION

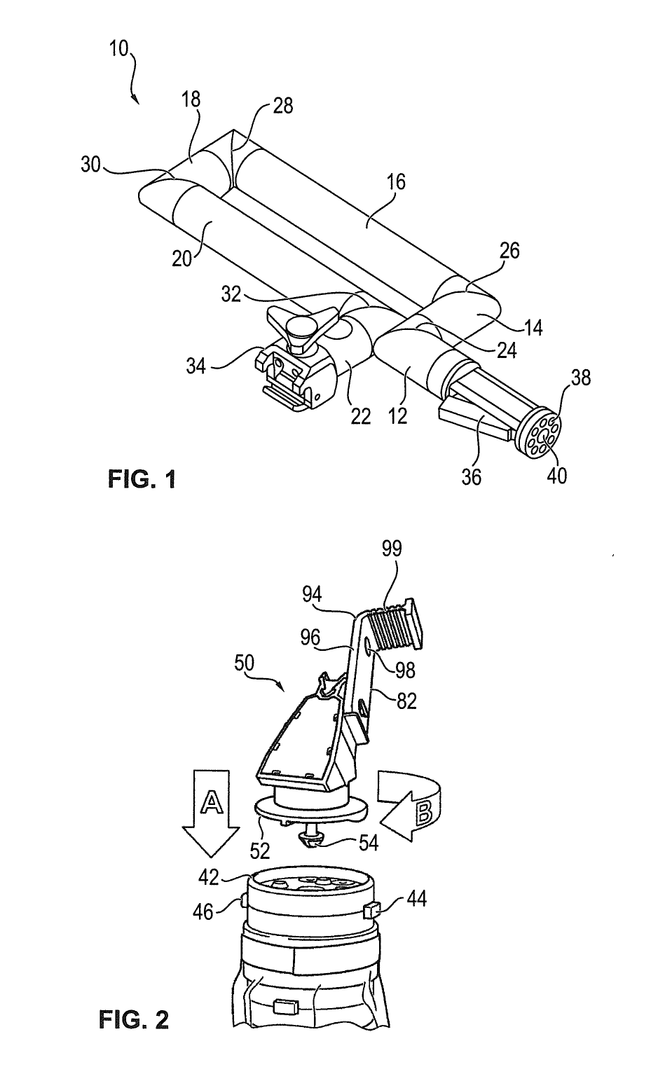

FIG. 1 shows a joint arm 10, which is part of an assistance system according to the invention.

The joint arm 10 includes a plurality of rigid holding members 12, 14, 16, 18, 20 and 22 which are coupled via joints 24, 26, 28, 30 and 32 to each other. At one end of the joint arm 10 a mounting device 34 is arranged, which serves to attach the joint arm 10 to a not shown slide rail of an operating table. At the other end of the joint arm 10 a handle 36 is positioned, which can be manually operated by the user in order to unlock with the joint arm 10.

If no operating force is exerted on the handle 36, the holding members 12, 14, 16, 18, 20 and 22 of the joint arm 10 are rigidly coupled to each other via the joints 24, 26, 28, 30 and 32. In this state, the joint arm forms a rigid unit.

If the user presses the handle 36, the holding members 24, 26, 28, 30 and 32, which are coupled to each other via the joints 24, 26, 28, 30 and 32, become movable relative to each other via an unlocking mechanism, which is not part of the present invention and therefore is not described in more detail here, so that the user can orient the joint arm 10 in space as desired. If the user subsequently releases the handle 36 again, the joints 24 to 32 are locked and the joint arm 10 is fixed in its changed orientation.

FIG. 2 shows an instrument holder 50, which is part of the assistance system according to the invention. In order to couple the instrument holder 50 to the joint arm 10 a quick coupling 42 is provided which is put with one end on a locking plug 40, which is arranged at the handle 36 (cf. FIG. 1). The quick coupling 42 has two unlocking buttons 44 and 46, which are pressed in order to detach the quick coupling 42 from the joint arm 10.

The instrument holder 50 is adapted to the quick coupling 42. For this, the instrument holder 50 includes a coupling device 52 with a further locking plug 54, which can be put into the quick coupling 42.

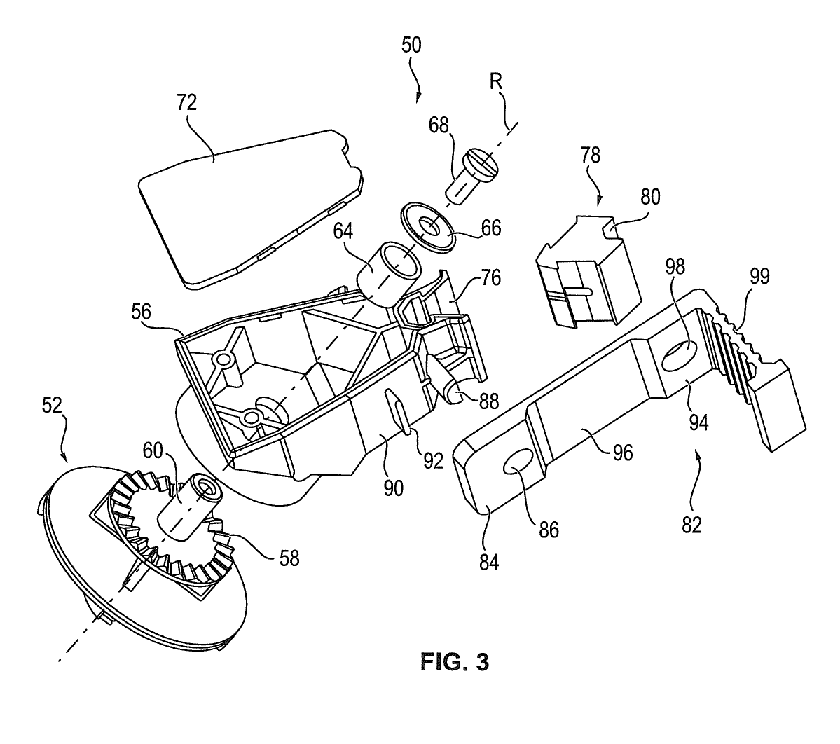

The instrument holder 50 is shown in FIG. 3 in an exploded view.

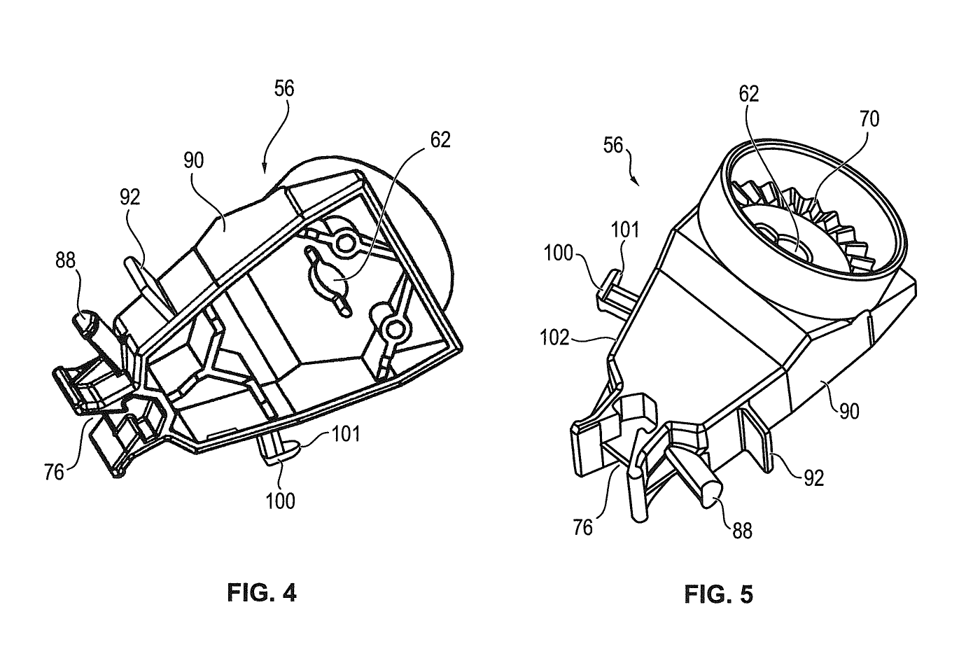

The instrument holder 50 includes as basic component a housing 56 produced in form of an integral plastic injection-moulded article, which is illustrated in different perspectives in FIGS. 4 and 5.

As shown in the exploded view of FIG. 3, when assembling the instrument holder 50 the coupling device 52 is attached to the housing 56. The coupling device 52 is formed from a circular locking plate, which includes a first circumferential toothing 58 on its side facing the housing 56. Inside of the toothing 58 a threaded shank 60 is positioned, which is inserted into a mounting opening 62 (cf. FIG. 4) formed in the housing 56. If the threaded shank 60 is inserted in the mounting opening 62, a compression coil spring 64 and a washer 66 are successively put on the threaded shank 60. Subsequently, a screw 68 is screwed in the threaded shank 60, thus tightening the locking plate 52 against the spring force exerted by the compression coil spring 64 on the housing 56.

As shown in FIG. 5, the housing 56 includes a second toothing 70, which surrounds the mounting hole 62. The second toothing 70 of the housing 56 is engaged with the first toothing 58 formed on the locking plate 52. The locking plate 52 can be lifted from the housing 56 so far against the spring force exerted by the compression coil spring 64 that the first toothing 58 formed on the locking plate 52 is detached from the first toothing 70 formed in the housing 56. In this state, the housing 56 can be rotated about a rotational axis R (cf. FIG. 3), which is coincident with the center axis of the threaded shank 60, relative to the locking plate 52. In this manner, the instrument holder 50 can be rotated relative to the quick coupling 42 and thus to the joint arm 10. A lid 72 closes the housing 56.

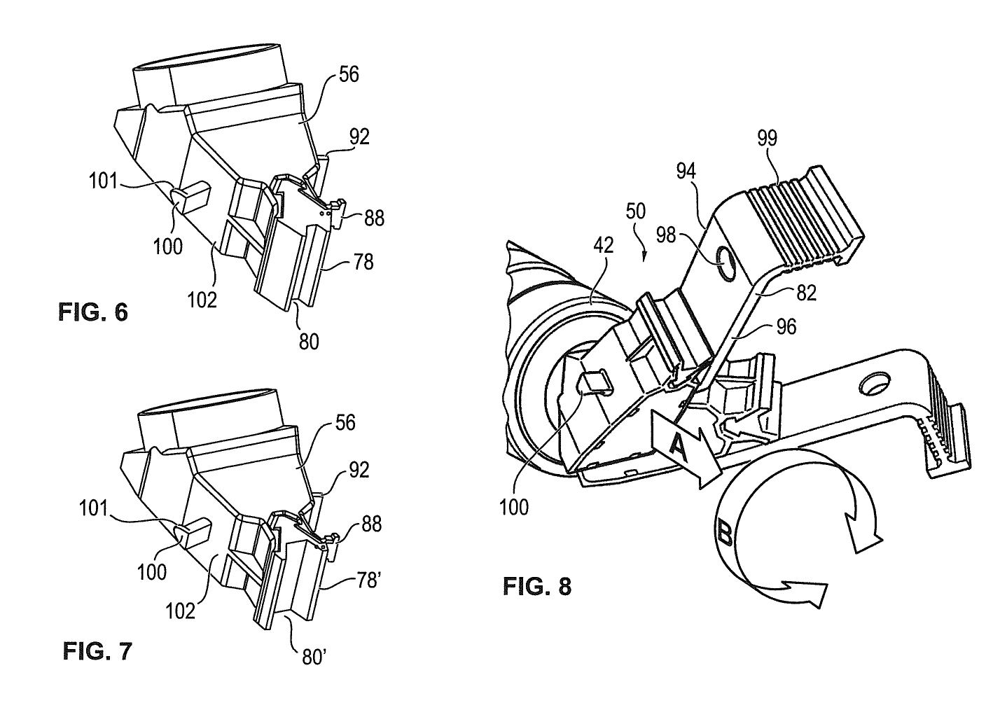

The housing 56 includes at its end facing away from the locking plate 52 a fitting opening 76, in which an elastic receiving piece 78 is inserted. The receiving piece 78 is e.g. produced from rubber or silicon and rests positively in the fitting opening 76. As can best be seen in in the illustration according to FIG. 6, the receiving piece 78 and the fitting opening 76 engage positively in a dovetail joint manner.

The receiving piece 78 includes on its side facing away from the fitting opening 76 a receiving groove 80 being trapezoid in cross section, which serves to receive a part of the medical instrument, which shall be held with the instrument holder 50. Correspondingly, shape and size of the receiving groove 80 are adapted to this instrument part.

As can be seen from the illustrations according to FIGS. 6 and 7, different elastic receiving pieces 78, 78′ can be provided, the respective receiving groove 80, 80′ of which is adapted to the medical instrument just being used. For this, said parts of the receiving pieces 78, 78′, which are adapted to the fitting opening 76 and form the dovetail joint therewith, are formed identically. Only the receiving grooves 80, 80′ are different.

The instrument holder 50 further includes an elastic pull strap 82, which serves to clamp the instrument part positioned in the receiving groove 80 of the mounting piece 78 in the receiving groove 80. The pull strap 82, which is e.g. produced from rubber or silicon, has a first strap portion 84, in which a mounting hole 86 is formed. The first strap portion 84 is put with its mounting hole 86 on a mounting projection 88, so that the first strap portion 84 is fastened at an outer wall 90 of the housing 56. For this, the mounting projection 88 is so broad that the mounting hole 86, when it is put on the mounting projection 88, is elastically deformed causing the first strap portion 84 to be fixed on the mounting projection 88. In this fixed state, the end surface of the first strap portion 84 abuts an abutment projection 92, which projects perpendicularly from the housing wall 90 of the housing 56.

The pull strap 82 has a second strap portion 94 and a third strap portion 96, which is arranged between the first strap portion 84 and the second strap portion 94. In the second strap portion 94 a second mounting hole 98 is formed which is put on a second mounting projection 100 (cf. FIGS. 4 and 5). The second mounting projection 100 has a locking nose 101, which points away from the receiving groove 80. It is positioned at a housing wall 102, which faces away from the housing wall 90, at which the first mounting projection 88 is formed. The two mounting projections 88 and 100 are thus positioned on different sides of the receiving groove 80.

The second strap portion 94 is put with its mounting hole 98 on the mounting projection 100 in order to attach the pull strap 82 to the housing 56 such that said pull strap 82 surrounds with its third strap portion 96 the receiving groove 80 formed in the mounting piece 78 together with the instrument part held therein and is thus pulled apart elastically in its longitudinal direction. Due to the elastic deformation of the pull strap 82 the third strap portion 96 surrounding the receiving groove 80 presses the instrument part into the receiving groove 80.

The pull strap 82 further has a fourth strap portion 99, which is adjacent to the second strap portion 94 at a right angle. The fourth strap portion 99 has a grooved surface and forms a handle portion, at which the user can grab the pull strap 82.

As mentioned above, the two mounting projections 88 and 100 are positioned on different sides of the fitting opening 76 and thus of the receiving groove 80 formed in the mounting piece 78. The distance, along which the pull strap 82 extends starting from the first mounting projection 88 over the receiving groove 80 up to the second mounting projection 100, is shorter than the distance between the two mounting holes 86 and 98 in the untensioned state of the pull strap 82. This ensures that the pull strap 82 in the tensioned state, in which the first mounting projection 88 engages the first mounting hole 86 and the second mounting projection 100 engages the second mounting hole 98, is sufficiently expanded in order to press the instrument part with a sufficiently great holding force into the receiving groove 80.

Below, the operating mode of the instrument holder 5 is explained with reference to the FIGS. 2, 8, 9 and 10.

For attaching the instrument holder 50 to the quick coupling 40 connected to the joint arm 10 the locking plug 54, which is formed on the locking plate 52, is put in the direction of arrow A (cf. FIG. 2) into the quick coupling 42. When the instrument holder 50 is subsequently rotated in the direction of arrow B, it engages audibly and is locked with the quick coupling 42.

FIG. 8 shows how the locked instrument holder 50 can be rotated relative to the quick coupling 42 in order to attach the instrument holder 50 for example to an already oriented endoscope. For this, the housing 56 is lifted a little from the locking plate 52 in the direction of arrow A against the spring force exerted by the compression coil spring 64, so that the two toothings 58 and 70 disengage. In this disengaged state, the housing 56 can be rotated relative to the locking plate 52, as indicated by arrow B in FIG. 8. To illustrate this process the instrument holder 50 is shown in two rotation positions in FIG. 8.

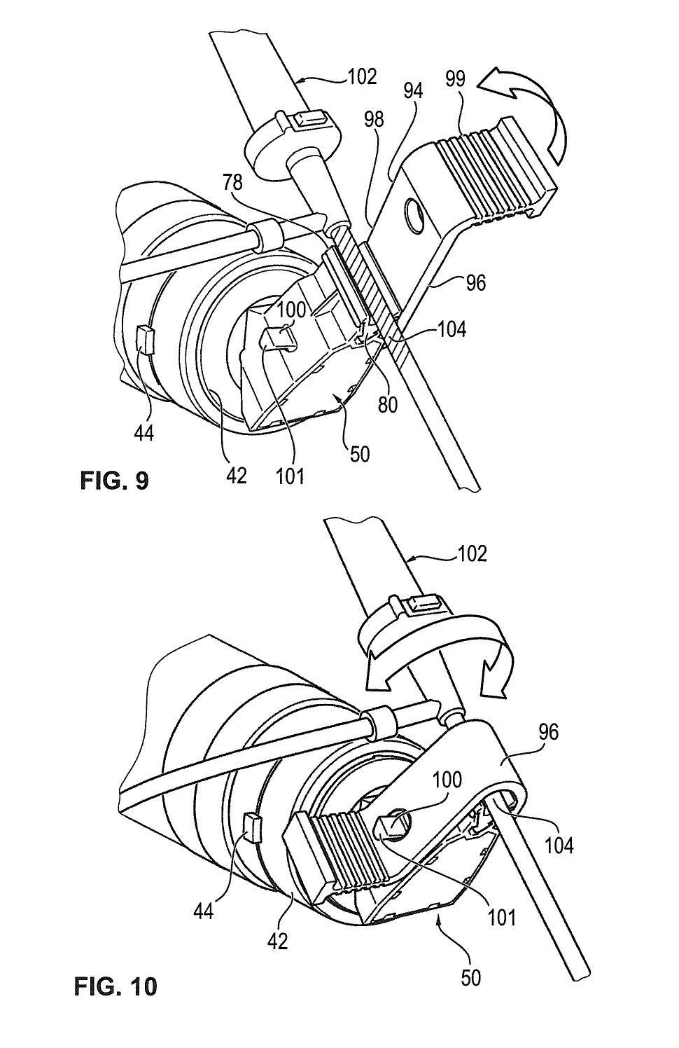

In FIG. 9 it is illustrated how an endoscope 102 is attached to the instrument holder 50. For this, a tube portion 104 of the endoscope 102 is positioned in the receiving groove 80 of the mounting piece 78. Subsequently, the operator grabs the grooved handle portion 99 of the pull strap 82 and turns the pull strap 82 down, as indicated by the arrow in FIG. 9.

Finally, the user clamps the tube portion 104 of the endoscope 102 in the receiving groove 80 by hooking the mounting projection 100 in the mounting hole 98 of the pull strap 82, as shown in FIG. 10. The illustration according to FIG. 10 also indicates that the endoscope 102 can be rotated about its longitudinal axis, although it is held in the receiving groove 80 by the pull strap 82. The pull strap 82 thus allows a limited rotational movement of the endoscope 102 arranged in the receiving groove 80 without having to detach the pull strap 82. In static operation, in which no external force acts on the endoscope, the latter is thus held securely in the receiving groove 80 by the pull strap 82. In dynamic operation, in which the user exerts a rotary force on the endoscope 102, the pull strap 82, on the other hand, allows a rotational movement of the endoscope 102 about its longitudinal axis. This is in particular advantageous when using angular optics. An active detachment of the endoscope, such as it is for example intended with customary screw clamps, is thus not required.

Although various embodiments of the present invention have been described and shown, the invention is not restricted thereto, but may also be embodied in other ways within the scope of the subject-matter defined in the following claims.

What is claimed is:

1. An instrument holder for mounting a medical instrument on a joint arm, comprising:
a housing,
a holder formed on the housing in order to hold the medical instrument, and
a coupling device formed on the housing in order to attach the housing to the joint arm,
wherein the holder has a receiving groove adapted to a part of the medical instrument, in which receiving groove said instrument part is receivable, and an elastic pull strap that is attachable to the housing in such a manner that it surrounds the receiving groove together with the instrument part arranged in the receiving groove in order to hold the instrument.

2. The instrument holder according to claim 1, wherein the pull strap has a first strap portion, which is attached on one side of the receiving groove, and a second strap portion, which is mountable on the other side of the receiving groove on the housing under elastic expansion of the pull strap, whereby a third strap portion positioned between the first and the second strap portion surrounds the receiving groove in an elastically expanded state.

3. The instrument holder according to claim 2, wherein the second strap portion is mountable via a pin/hole-connection on the housing.

4. The instrument holder according to claim 3, wherein the second strap portion has a mounting hole and the housing has a mounting projection engaging the mounting hole, which mounting projection includes a locking nose at its free end, which locking nose points away from the receiving groove.

5. The instrument holder according to that claim 2, wherein the first, the second and the third strap portion form a straight, elongated element in the untensioned state of the pull strap.

6. The instrument holder according to claim 5, wherein the pull strap has a fourth strap portion adjacent to the second strap portion, which extends at an angle from the straight element in the untensioned state of the pull strap.

7. The instrument holder according to that claim 1, wherein the pull strap is formed from an elastomer.

8. The instrument holder according to that claim 1, wherein the pull strap holds the instrument part in such a manner in the receiving groove that the instrument part is rotatable about its longitudinal axis against the holding force exerted by the pull strap.

9. The instrument holder according to that claim 1, wherein the receiving groove is formed in an elastic receiving piece, which rests in a fitting opening formed in the housing.

10. The instrument holder according to claim 9, wherein the receiving piece and the fitting opening engage in a dovetail joint manner.

11. The instrument holder according to claim 1, wherein the receiving piece is formed from an elastomer.

12. The instrument holder according to claim 1, wherein the housing is rotatable relative to the coupling device about a rotational axis (R).

13. The instrument holder according to claim 12, wherein the coupling device comprises a locking plate having a first toothing, which is in locking engagement with a second toothing formed on the housing, wherein the second toothing is detachable against the biasing force exerted by a biasing element along the rotational axis (R) from the first toothing, in order to rotate the housing relative to the locking plate plate about the rotational axis (R).

14. The instrument holder according to claim 13, wherein the locking plate is mounted on the housing by means of a screwed connection, which is formed from a threaded shank and a screw element inserted into the threaded shank, and is pressed against the housing by means of a compression spring, in which the threaded shank is inserted.

15. A joint arm system, comprising a joint arm, a medical instrument, preferably an endoscope, and an instrument holder according to claim 1 for mounting the medical instrument on the joint arm.

16. The instrument holder according to claim 3, wherein the first, the second and the third strap portion form a straight, elongated element in the untensioned state of the pull strap.

17. The instrument holder according to claim 4, wherein the first, the second and the third strap portion form a straight, elongated element in the untensioned state of the pull strap.

18. The instrument holder according to claim 2, wherein the pull strap is formed from an elastomer.

19. The instrument holder according to claim 3, wherein the pull strap is formed from an elastomer.

20. The instrument holder according to claim 4, wherein the pull strap is formed from an elastomer.
